(19) United States
(12) Patent Application Publication
(10) Pub. No.: US 2024/0013888 A1
(43) Pub. Date: Jan. 11, 2024

(54) BODY SHAPE MANAGEMENT SYSTEM, ITS CONTROL METHOD, AND CONTROL PROGRAM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Narimasa WATANABE, Chofu-shi (JP); Kohei OTSUKA, Sagamihara-shi (JP); Ryusuke KURODA, Edogawa-ku (JP)

(73) Assignee: TOYOTA JIDOSHA KABUSHIKI KAISHA, Toyota-shi (JP)

(21) Appl. No.: 18/344,762

(22) Filed: Jun. 29, 2023

(30) Foreign Application Priority Data
Jul. 5, 2022 (JP) 2022-108253

(51) Int. Cl. G16H 20/30; G16H 20/60

(52) U.S. Cl. CPC G16H 20/30; G16H 20/60

(57) ABSTRACT

A body shape management system includes: an acquisition unit configured to acquire information about an actual body shape of a user and information about a target body shape of the user that he/she wants to have after a lapse of a set period; a display control unit configured to display at least an avatar representing the target body shape of the user acquired by the acquisition unit on a monitor; a plan calculation unit configured to calculate at least one plan for a behavioral item that the user should perform during the set period based on the actual body shape of the user and the target body shape of the user that he/she wants to have after the lapse of the set period; and an output unit configured to output the at least one plan calculated by the plan calculation unit.

### CROSS REFERENCE TO RELATED APPLICATIONS

This application is based upon and claims the benefit of priority from Japanese patent application No. 2022-108253, filed on Jul. 5, 2022, the disclosure of which is incorporated herein in its entirety by reference.

### BACKGROUND

The present disclosure relates to a body shape management system, its control method, and a control program.

Japanese Unexamined Patent Application Publication No. 2007-310632 discloses an avatar health and body shape prediction system capable of displaying an avatar of a user and predicting his/her own body shape and health condition that he/she will have after a lapse of an arbitrary period.

### SUMMARY

However, regarding Japanese Unexamined Patent Application Publication No. 2007-310632, there is a problem that it does not propose any specific exercise and meal plan for making the body shape of a user closer to his/her target body shape.

The present disclosure has been made in view of the above-described circumstances, and an object thereof is to provide a body shape management system, its control method, and a control program capable of proposing a behavioral plan suitable for a user.

A body shape management system according to the present disclosure includes: an acquisition unit configured to acquire information about an actual body shape of a user and information about a target body shape of the user that he/she wants to have after a lapse of a set period; a display control unit configured to display at least an avatar representing the target body shape of the user acquired by the acquisition unit on a monitor; a plan calculation unit configured to calculate at least one plan for a behavioral item that the user should perform during the set period based on the actual body shape of the user and the target body shape of the user that he/she wants to have after the lapse of the set period; and an output unit configured to output the at least one plan calculated by the plan calculation unit. The above-described body shape management system can visually express a target body shape of a user by using an avatar, and thereby can propose, to the user, a behavioral plan suitable for him/her in a user-friendly manner.

The display control unit may display the avatar representing the actual body shape of the user on the monitor in such a manner that the avatar can be edited, and the acquisition unit may acquire the information about the target body shape of the user that he/she wants to have after the lapse of the set period based on the editing made to the avatar representing the actual body shape of the user displayed on the monitor.

The acquisition unit may be further configured to acquire, in addition to the information about the actual body shape of the user, a predetermined plan for the behavioral item that the user should perform during the set period; when the predetermined plan is acquired by the acquisition unit, the display control unit may display an avatar representing a predicted body shape of the user on the monitor in such a manner that the avatar can be edited, the predicted body shape being a body shape which it is predicted the user will have after the lapse of the set period on a condition that the user behaves according to the predetermined plan; and the acquisition unit may further acquire the information about the target body shape of the user that he/she wants to have after the lapse of the set period based on the editing made to the avatar representing the predicted body shape of the user displayed on the monitor.

The plan calculation unit may give preference to incorporating an exercise item and a meal included in the predetermined plan into the calculated plan over an exercise item and a meal that are not included in the predetermined plan.

The body shape management system may further include a database in which a schedule of the user during the set period is stored, in which the plan calculation unit may calculate at least one plan for a behavioral item that the user should perform during the set period based on, in addition to the actual body shape of the user and the target body shape of the user that he/she wants to have after the lapse of the set period, the schedule of the user during the set period stored in the database.

The display control unit may further display the avatar representing the actual body shape of the user on the monitor.

The display control unit may superimpose the avatar representing the actual body shape of the user on the avatar representing the target body shape of the user on the monitor.

The display control unit may display the avatar representing the actual body shape of the user and the avatar representing the target body shape of the user in an alternate manner.

The body shape management system may further include a predicted body shape calculation unit configured to calculate the predicted body shape of the user after the lapse of the set period according to a change in the actual body shape of the user acquired by acquisition unit, in which the display control unit may further display the avatar representing the predicted body shape of the user calculated by predicted body shape calculation unit on the monitor.

The display control unit may superimpose the avatar representing the predicted body shape of the user on the avatar representing the target body shape of the user on the monitor.

The display control unit may display the avatar representing the predicted body shape of the user and the avatar representing the target body shape of the user in an alternate manner.

The plan calculation unit may recalculate at least one plan for a behavioral item that the user should perform during the set period based on a difference between the predicted body shape and the target body shape.

The output unit may display the at least one plan calculated by the plan calculation unit on the monitor.

The output unit may display a plan selected from the at least one plan calculated by the plan calculation unit along with the schedule of the user on the monitor.

A method for controlling a body shape management system according to the present disclosure includes: acquiring information about an actual body shape of a user and information about a target body shape of the user that he/she wants to have after a lapse of a set period; displaying at least an avatar representing the acquired target body shape of the user on a monitor; calculating at least one plan for a behavioral item that the user should perform during the set period based on the actual body shape of the user and the target body shape of the user that he/she wants to have after the lapse of the set period; and outputting the calculated at least one plan. By the above-described method for controlling a body shape management system, it is possible to visually express a target body shape of a user by using an avatar, and thereby to propose, to the user, a behavioral plan suitable for him/her in a user-friendly manner.

A control program according to the present disclosure causes a computer to perform: a process for acquiring information about an actual body shape of a user and information about a target body shape of the user that he/she wants to have after a lapse of a set period; a process for displaying at least an avatar representing the acquired target body shape of the user on a monitor; a process for calculating at least one plan for a behavioral item that the user should perform during the set period based on the actual body shape of the user and the target body shape of the user that he/she wants to have after the lapse of the set period; and a process for outputting the calculated at least one plan. By the above-described control program, it is possible to visually express a target body shape of a user by using an avatar, and thereby to propose, to the user, a behavioral plan suitable for him/her in a user-friendly manner.

According to the present disclosure, it is possible to provide a body shape management system, its control method, and a control program capable of proposing a behavioral plan suitable for a user.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing an example of a configuration of a body shape management system according to a first embodiment;

FIG. 2 is a flowchart showing operations performed by the body shape management system according to the first embodiment;

FIG. 3 shows an example of an avatar of a user displayed on a monitor;

FIG. 4 shows an example of avatars of a user displayed on a monitor;

FIG. 5 shows an example of avatars of a user displayed on a monitor;

FIG. 6 shows an example of avatars of a user displayed on a monitor;

FIG. 7 shows an example of a plurality of behavioral plans displayed on a monitor;

FIG. 8 is a block diagram showing an example of a configuration of a body shape management system according to a second embodiment;

FIG. 9 shows an example of a behavioral plan displayed on a monitor along with a schedule of a user;

FIG. 10 is a block diagram showing an example of a configuration of a body shape management system according to a third embodiment; and

FIG. 11 shows an example of avatars of a user displayed on a monitor.

### DESCRIPTION OF EMBODIMENTS

The present disclosure will be described hereinafter through embodiments of the disclosure, but the invention according to the claims is not limited to the below-shown embodiments. Further, all the components/structures described in the embodiments are not necessarily indispensable as means for solving the problem. For clarifying the explanation, the following description and the drawings are partially omitted and simplified as appropriate. The same reference numerals (or symbols) are assigned to the same elements throughout the drawings and redundant descriptions thereof are omitted as appropriate.

### First Embodiment

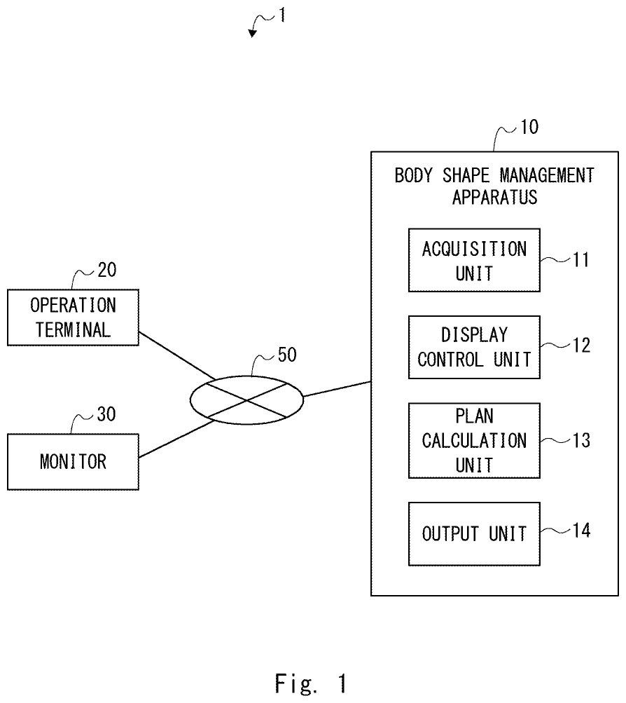

FIG. 1 is a block diagram showing an example of a configuration of a body shape management system 1 according to a first embodiment. The body shape management system 1 can visually express a target body shape of a user by using an avatar (a virtual person), and thereby can propose, to the user, a behavioral plan suitable for him/her in a user-friendly manner as well as encouraging the user to achieve his/her target. Details of the body shape management system 1 or the like will be described hereinafter.

As shown in FIG. 1, the body shape management system 1 includes a body shape management apparatus 10, an operation terminal 20, a monitor 30, and a network 50. The body shape management apparatus 10 can also be referred to as a body shape management system. The operation terminal 20, the monitor 30 and the body shape management apparatus 10 are configured so that they can communicate with each other through the wired or wireless network 50.

The operation terminal 20 is, for example, a PC (Personal Computer) terminal, a mobile terminal such as a smartphone or a tablet-type terminal, or a dedicated communication terminal provided for the body shape management system. For example, a user U1 enters information about the actual body shape of the user U1 and information about a target body shape of the user U1 that he/she wants to have after a lapse of a set period into the operation terminal 20 by operating an operation screen, a keyboard, or the like of the operation terminal 20. The operation terminal 20 receives the information about the actual body shape of the user U1 and the information about the target body shape of the user U1 that he/she wants to have after the lapse of the set period, entered by the operation performed by the user U1, and transmits the received information items to the body shape management apparatus 10 through the network 50.

Note that the information about the actual body shape of the user U1 means information about the apparent body shape of the user U1 at the present time (at the time when the information is entered). The information about the actual body shape of the user U1 may be directly entered by the user U1, or may be estimated by the body shape management apparatus 10 or the like based on information about the user U1, for example, his/her age, gender, height, and weight at the present time.

Further, the information about the target body shape of the user U1 that he/she wants to have after the lapse of the set period is information about a target apparent body shape of the user U1 that he/she wants to have after the lapse of the setting period. The set period can be arbitrarily set by the user U1, and can be set to, for example, one month, three months, or one year. The information about the target body shape of the user U1 that he/she wants to have after the lapse of the set period may be directly entered by the user U1, or may be estimated by the body shape management apparatus 10 or the like based on information about, for example, a target weight and/or a target body fat percentage that he/she wants to have after the lapse of the set period. A specific example as to how to enter information about the target body shape of the user U1 that he/she wants to have after the lapse of the set period will be described later.

Note that the operation terminal 20 may receive information about a predetermined behavioral plan desired by the user U1 in addition to the information about the actual body shape of the user U1 and the information about the target body shape of the user U1 that he/she wants to have after the lapse of the set period, and transmit the received information items to the body shape management apparatus 10 through the network 50.

The monitor 30 is, for example, a monitor of a PC terminal, a monitor of a mobile terminal such as a smartphone or a tablet-type terminal, or a dedicated monitor prepared for the body shape management system. The monitor 30 receives results output from the body shape management apparatus 10 (which will be described later) through the network 50, and displays the received results.

Note that the monitor 30 may be integrated into the operation terminal 20 or provided separately from the operation terminal 20. In this embodiment, an example case where the operation terminal 20 is a PC terminal and the monitor 30 is integrated into the operation terminal will be described. That is, in this embodiment, an example case where the monitor 30 is used as the monitor of the operation terminal 20, which is a PC terminal, will be described. Therefore, in addition to the results output from the body shape management apparatus 10, input items and the like for information to be transmitted to the body shape management apparatus 10 (i.e., information about the actual body shape of the user U1 and information about the target body shape of the user U1 that he/she wants to have after the lapse of the set period) may be displayed on the monitor 30. In this case, the user U1 enters necessary information in the input items displayed on the monitor 30 by operating the keyboard and/or the mouse of the operation terminal 20, which is a PC terminal, and thereby enters information about the actual body shape of the user U1 and information about the target body shape of the user U1 that he/she wants to have after the lapse of the set period into the operation terminal 20. The operation terminal 20 receives these information items entered by the operation performed by the user U1, and transmits the received information items to the body shape management apparatus 10 through the network 50.

The body shape management apparatus 10 displays an avatar (a virtual person) representing the target body shape of the user U1 on the monitor 30, calculates at least one plan (at least one behavioral plan) for behavioral items that the user U1 should perform during the set period based on the actual body shape of the user U1 and the target body shape of the user U1 that he/she wants to have after the lapse of the set period, and outputs the calculated at least one plan.

Specifically, the body shape management apparatus 10 includes at least an acquisition unit 11, a display control unit 12, a plan calculation unit 13, and an output unit 14.

The acquisition unit 11 acquires information entered by the user U1 through the operation terminal 20. That is, the acquisition unit 11 acquires, for example, information about the actual body shape of the user U1 and information about the target body shape of the user U1 that he/she wants to have after the lapse of the set period. Note that the information acquired by the acquisition unit 11 is not limited to the above-described information. That is, the acquisition unit 11 may acquire other information used for the calculation of a behavioral plan performed by the plan calculation unit 13 (which will be described later).

The display control unit 12 displays at least an avatar representing the target body shape of the user U1 that he/she wants to have after the lapse of the set period, acquired by the acquisition unit 11 on the monitor Note that the expression that the display control unit 12 displays the avatar of the user U1 on the monitor 30 may include processes in which the display control unit 12 outputs the avatar of the user U1 and the monitor 30 acquires and displays the avatar of the user U1 output from the display control unit 12. In this way, the user U1 can visually know his/her target body shape, so that it is possible to encourage the user U1 to achieve the target.

Note that the display control unit 12 may also display an avatar representing the actual body shape of the user U1 as well as the avatar representing the target body shape of the user U1 on the monitor 30. In this way, the user U1 can visually know a difference between his/her actual body shape and his/her target body shape. Note that the display control unit 12 may superimpose the avatar representing the actual body shape of the user U1 on the avatar representing the target body shape of the user U1 on the monitor 30, or display the avatar representing the actual body shape of the user U1 and the avatar representing the target body shape of the user U1 in an alternate manner.

Further, the display control unit 12 may display the avatar representing the actual body shape of the user U1 on the monitor 30 in such a manner that the avatar can be edited. For example, the user U1 edits the avatar representing the actual body shape of the user U1 by operating the operation terminal 20, and by doing so, forms an avatar representing the target body shape of the user U1. Note that the acquisition unit 11 can acquire information about the target body shape of the user U1 that he/she wants to have after the lapse of the set period based on the editing made to the avatar representing the actual body shape of the user U1 (e.g., based on the degree of reduction in waistline).

Further, when information about a predetermined behavioral plan desired by the user U1 is acquired by the acquisition unit 11, the display control unit 12 may display an avatar representing a predicted body shape of the user U1, which is a body shape which it is predicted the user U1 will have after the lapse of the set period on the condition that the user U1 behaves according to the predetermined behavioral plan, on the monitor 30 in such a manner that the avatar can be edited. In this way, the user U1 can visually know his/her predicted body shape that he/she will have when he/she behaves according to the predetermined desired behavioral plan. Note that when the predicted body shape of the user U1 differs from his/her target body shape, the user U1 edits the avatar representing the predicted body shape of the user U1 by operating the operation terminal 20, and by doing so, forms an avatar representing the target body shape of the user U1. Note that the acquisition unit 11 can acquire information about the target body shape of the user U1 that he/she wants to have after the lapse of the set period based on the editing made to the avatar representing the predicted body shape of the user U1.

The plan calculation unit 13 calculates at least one plan (at least one behavioral plan) for behavioral items that the user U1 should perform during the set period based on the actual body shape of the user U1 and the target body shape of the user U1 that he/she wants to have after the lapse of the set period, acquired by the acquisition unit 11. Note that the behavioral items include meals and exercise items.

For example, when the plan calculation unit 13 has compared the actual body shape of the user U1 and his/her target body shape, and determined that it is necessary to reduce the weight of the user U1 from 80 kg to 70 kg, it calculates at least one behavioral plan including meals and exercise items for reducing the weight from 80 kg to 70 kg by the end of the set period, such as regular jogging practices and meal menus with reduced calorie intake. Further, for example, when the plan calculation unit 13 has compared the actual body shape of the user U1 and his/her target body shape, and determined that it is necessary to reduce the waistline of the user U1 by 5 cm and/or reduce his/her upper arm circumference by 1 cm, it calculates at least one behavioral plan including meals and exercise items for reducing the waistline by 5 cm and reducing the upper arm circumference by 1 cm by the end of the set period, such as regular sit-up exercises and/or push-ups exercise. Further, for example, when the plan calculation unit 13 has compared the actual body shape of the user U1 and his/her target body shape, and determined that it is necessary to increase the chest circumference of the user U1 by 2 cm, it calculates at least one behavioral plan including meals and exercise items for increasing the chest circumference by 2 cm by the end of the set period, such as regular hard push-up exercises.

Note that in the case where prohibited foods or prohibited exercises are specified, the plan calculation unit 13 may calculate a behavioral plan consisting of meals that do not include any of the prohibited foods and exercise items other than the prohibited exercises.

Further, when a predetermined behavioral plan desired by the user U1 is acquired by the acquisition unit 11, the plan calculation unit 13 may give preference to incorporating exercise items and meals included in this predetermined behavioral plan into the calculated plan. For example, when running is included in the exercise items of the predetermined behavioral plan, the plan calculation unit 13 may preferentially calculate (i.e., preferentially make) a behavioral plan in which running is included in its exercise items.

The output unit 14 outputs at least one behavioral plan calculated by the plan calculation unit 13. Information about at least one behavioral plan output from the output unit 14 is transferred, for example, to the monitor 30 through the network 50, and displayed on the monitor 30. Note that the information about at least one behavioral plan output from the output unit 14 does not necessarily have to be displayed only on the monitor 30. That is, it may be output in the form of voice, or may be displayed on the monitor of the operation terminal 20 when the monitor 30 and the operation terminal 20 are separate apparatuses or the like.

### (Operation of Body Shape Management System 1)

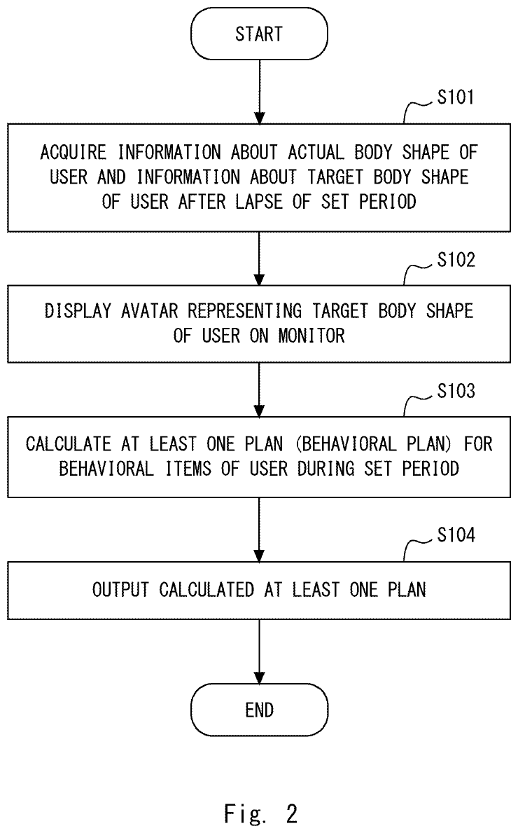

Next, operations performed by the body shape management system 1 will be described with reference to FIG. 2. FIG. 2 is a flowchart showing operations performed by the body shape management system 1.

Firstly, the operation terminal 20 receives information about the actual body shape of the user U1 and information about a target body shape of the user U1 that he/she wants to have after a lapse of a set period, entered by an operation performed by the user U1, and transmits the received information items to the body shape management apparatus through the network 50. Note that the operation terminal 20 may receive information about a predetermined behavioral plan desired by the user U1 in addition to the information about the actual body shape of the user U1 and the information about the target body shape of the user U1 that he/she wants to have after the lapse of the set period, and transmit the received information items to the body shape management apparatus through the network 50.

For example, the user U1 enters necessary information in input items displayed on the monitor 30 by operating the keyboard and/or the mouse of the operation terminal 20, which is a PC terminal, and thereby enters information about the actual body shape of the user U1 and information about the target body shape of the user U1 that he/she wants to have after the lapse of the set period into the operation terminal 20. The operation terminal 20 receives these information items entered by the operation performed by the user U1, and transmits the received information items to the body shape management apparatus 10 through the network 50.

In the body shape management apparatus 10, the acquisition unit 11 acquires information entered by the user U1 through the operation terminal 20. That is, the acquisition unit 11 acquires, for example, information about the actual body shape of the user U1 and information about the target body shape of the user U1 that he/she wants to have after the lapse of the set period (Step S101).

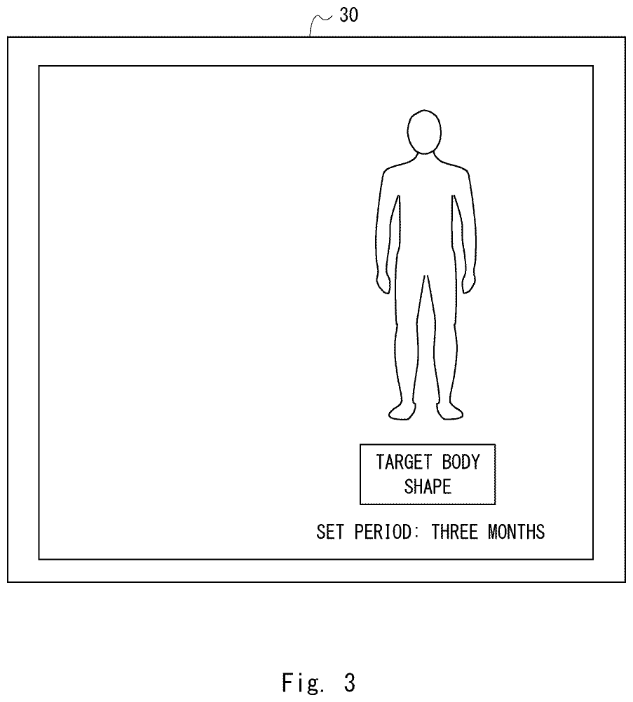

After that, in the body shape management apparatus 10, the display control unit 12 displays at least an avatar representing the target body shape of the user U1 that he/she wants to have after the lapse of the set period, acquired by the acquisition unit 11 on the monitor 30 (Step S102). FIG. 3 shows an example of an avatar representing a target body shape of the user U1 displayed on the monitor 30. In the example shown in FIG. 3, an avatar representing a target body shape of the user U1 in a case where the set period is three months is shown. In this way, the user U1 can visually know his/her target body shape, so that it is possible to encourage the user U1 to achieve the target.

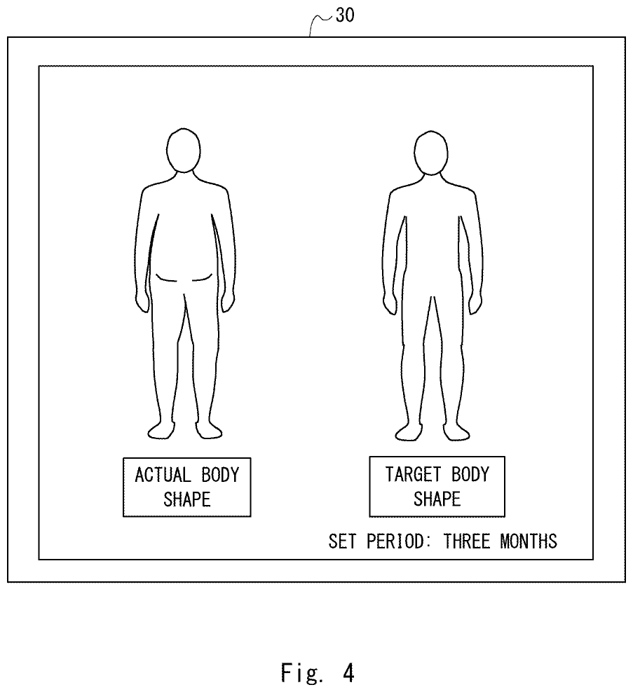

Note that the display control unit 12 may also display an avatar representing the actual body shape of the user U1 as well as the avatar representing the target body shape of the user U1 on the monitor 30. In this way, the user U1 can visually know a difference between his/her actual body shape and his/her target body shape. Note that the display control unit 12 may superimpose the avatar representing the actual body shape of the user U1 on the avatar representing the target body shape of the user U1 on the monitor 30, or display the avatar representing the actual body shape of the user U1 and the avatar representing the target body shape of the user U1 in an alternate manner. FIG. 4 shows an example of an avatar representing the actual body shape of the user U1 and an avatar representing the target body shape of the user U1 displayed on the monitor 30. In the example shown in FIG. 4, the avatar representing the actual body shape of the user U1 and the avatar representing the target body shape of the user U1 are displayed side by side.

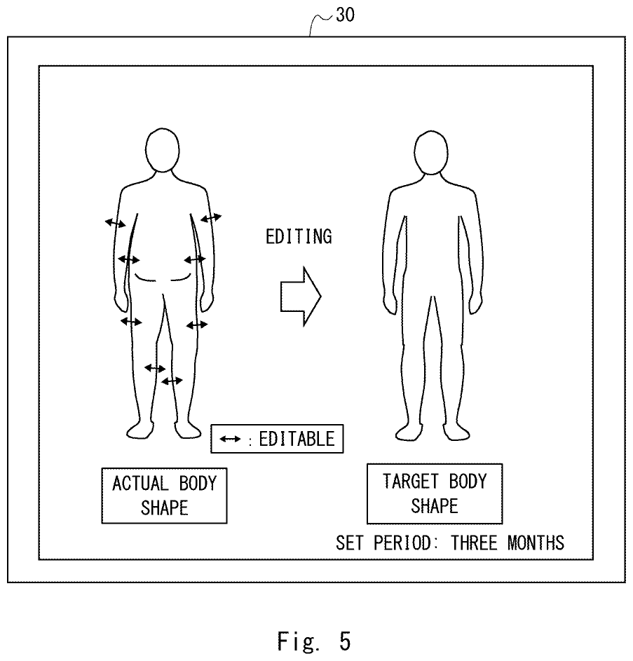

Further, the display control unit 12 may display the avatar representing the actual body shape of the user U1 on the monitor 30 in such a manner that the avatar can be edited. For example, the user U1 edits the avatar representing the actual body shape of the user U1 by operating the operation terminal 20, and by doing so, forms an avatar representing the target body shape of the user U1. Note that the acquisition unit 11 can acquire information about the target body shape of the user U1 that he/she wants to have after the lapse of the set period based on the editing made to the avatar representing the actual body shape of the user U1 (e.g., based on the degree of reduction in waistline). FIG. 5 shows an example of an avatar representing the actual body shape of the user U1 and an avatar representing the target body shape of the user U1 displayed on the monitor 30. In the example shown in FIG. 5, an avatar representing the target body shape of the user U1 that has been formed by editing the avatar representing the actual body shape of the user U1 is shown.

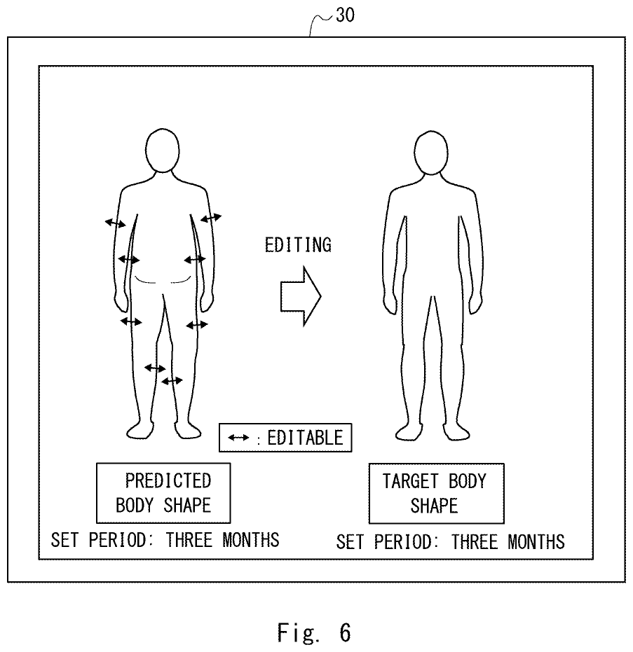

Further, when a predetermined behavioral plan desired by the user U1 is acquired by the acquisition unit 11, the display control unit 12 may display an avatar representing a predicted body shape of the user U1, which is a body shape which it is predicted the user U1 will have after the lapse of the set period on the condition that the user U1 behaves according to the predetermined plan, on the monitor 30 in such a manner that the avatar can be edited. In this way, the user U1 can visually know his/her predicted body shape that he/she will have when he/she behaves according to the predetermined desired behavioral plan. Note that when the predicted body shape of the user U1 differs from his/her target body shape, the user U1 edits the avatar representing the predicted body shape of the user U1 by operating the operation terminal 20, and by doing so, forms an avatar representing the target body shape of the user U1. Note that the acquisition unit 11 can acquire information about the target body shape of the user U1 that he/she wants to have after the lapse of the set period based on the editing made to the avatar representing the predicted body shape of the user U1. FIG. 6 shows an example of an avatar representing a predicted body shape of the user U1 and an avatar representing the target body shape of the user U1 displayed on the monitor 30. In the example shown in FIG. 6, an avatar representing the target body shape of the user U1 that has been formed by editing the avatar representing the predicted body shape of the user U1 is shown.

After that, in the body shape management apparatus 10, the plan calculation unit 13 calculates at least one plan (at least one behavioral plan) for behavioral items that the user U1 should perform during the set period based on the actual body shape of the user U1 and the target body shape of the user U1 that he/she wants to have after the lapse of the set period, acquired by the acquisition unit 11 (Step S103).

After that, in the body shape management apparatus 10, the output unit 14 outputs at least one behavioral plan calculated by the plan calculation unit 13 (Step S104). Information about at least one behavioral plan output from the output unit 14 is transferred, for example, to the monitor 30 through the network 50, and displayed on the monitor 30.

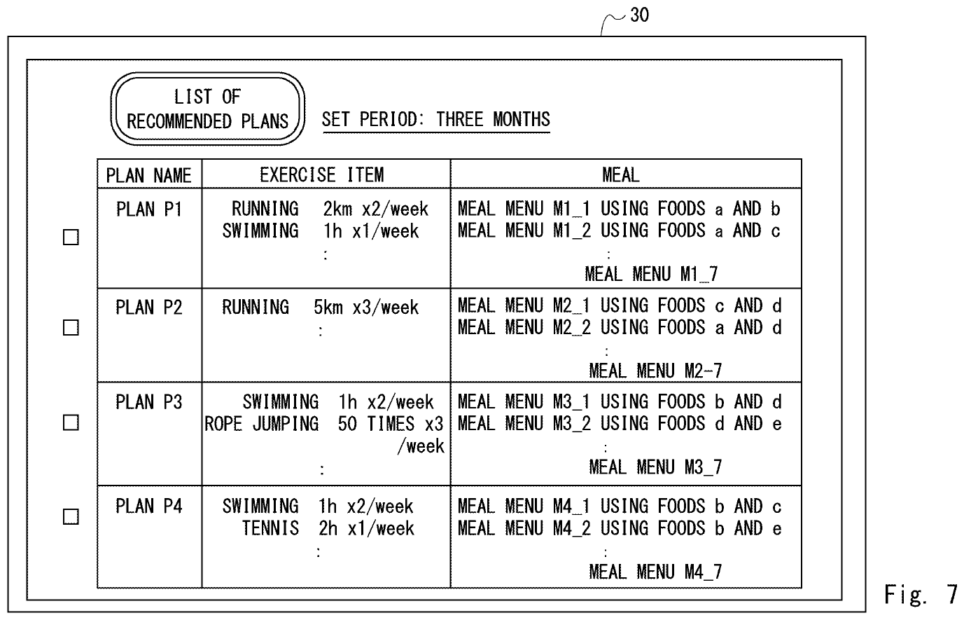

FIG. 7 shows an example of behavioral plans displayed on the monitor 30. Referring to FIG. 7, on the monitor 30, four behavioral plans P1 to P4 are displayed as behavioral plans (recommended plans) proposed to the user U1.

In the behavioral plan P1, it is proposed to incorporate, as exercise items, two 2 km runs and one one-hour swimming session in each week. Further, in the behavioral plan P1, it is proposed to incorporate, as meals, meal menus M1_1 to M1_7 including a meal menu M1_1 using foods a and b, and a meal menu M1_2 using foods a and c in each week.

In the behavioral plan P2, it is proposed to incorporate, as exercise items, three 5 km runs in each week. Further, in the behavioral plan P2, it is proposed to incorporate, as meals, meal menus M2_1 to M2_7 including a meal menu M2_1 using foods c and d, and a meal menu M2_2 using foods a and d in each week.

In the behavioral plan P3, it is proposed to incorporate, as exercise items, two one-hour swimming sessions and three 50 rope jumping sessions in each week. Further, in the behavioral plan P3, it is proposed to incorporate, as meals, meal menus M3_1 to M3_7 including a meal menu M3_1 using foods b and d, and a meal menu M3_2 using foods d and e in each week.

In the behavioral plan P4, it is proposed to incorporate, as exercise items, two one-hour swimming sessions and one two-hour tennis practices in each week. Further, in the behavioral plan P4, it is proposed to incorporate, as meals, meal menus M4_1 to M4_7 including a meal menu M4_1 using foods b and c, and a meal menu M4_2 using foods b and e in each week.

For example, the user U1 may adopt (i.e., choose) one of the behavioral plans P1 to P4 that he/she likes, and behaves while incorporating the meals and the exercise items proposed in the adopted behavioral plan. By doing so, the user U1 can make his/her actual body shape closer to his/her target body shape before the set period expires. That is, the body shape management system 1 can propose an appropriate behavioral plan for making the actual body shape of the user U1 closer to his/her target body shape before the set period expires.

Note that more specific items or the like may be presented (i.e., shown) as exercise items and meals included in each behavioral plan. For example, instead of the “one-hour swimming session”, “20 times of 50 m crawls” or the like may be presented in a more specific manner. Alternatively, for example, instead of the “one-hour gym training session”, “three sets of 10 times of 30 kg bench press, three sets of 10 times of 50 kg leg press, . . . .” or the like may be presented in a more specific manner.

As described above, the body shape management system 1 according to this embodiment can visually express a target body shape of the user U1 by using an avatar, and thereby can propose, to the user U1, a behavioral plan suitable for him/her in a user-friendly manner as well as encouraging the user U1 to achieve his/her target.

### Second Embodiment

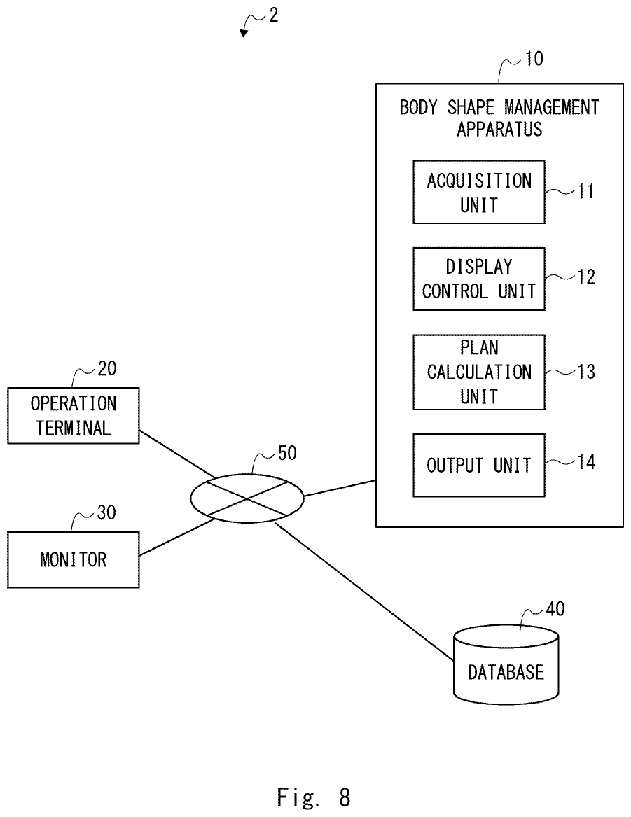

FIG. 8 is a block diagram showing an example of a configuration of a body shape management system 2 according to a second embodiment. The body shape management system 2 according to this embodiment is configured to calculate and propose a behavioral plan for a user U1 while also taking a work schedule of the user U1 into consideration.

As shown in FIG. 8, the body shape management system 2 further includes a database 40 as compared with the body shape management system 1. Information about the schedule of the user U1 is stored in the database 40. The rest of the configuration of the body shape management system 2 is similar to that of the body shape management system 1, and therefore the description thereof is omitted.

In the body shape management apparatus 10, the plan calculation unit 13 calculates at least one plan (at least one behavioral plan) for behavioral items that the user U1 should perform during a set period based on the schedule of the user U1 during the set period stored in the database 40 in addition to the actual body shape of the user U1 and the target body shape of the user U1 that he/she wants to have after the lapse of the set period. For example, the plan calculation unit 13 calculates a behavioral plan while also taking, for example, a spare time of the user U1, calories that will be taken in a dinner with a client or the like, and/or calories that will be consumed by commuting, a physical work, or the like into consideration. The behavioral plan calculated in the above-described calculation is displayed, for example, on the monitor 30 along with the schedule of the user U1.

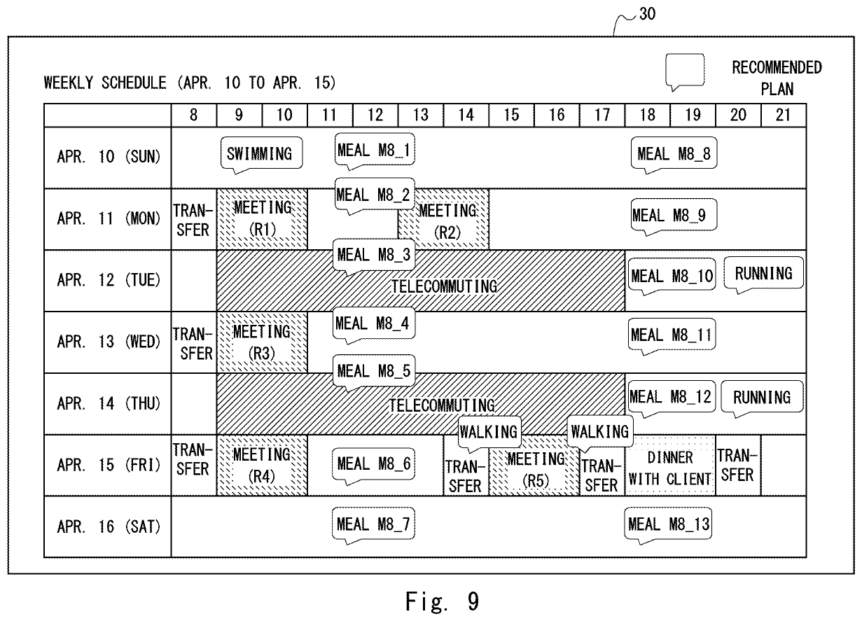

FIG. 9 shows an example of a behavioral plan of the user U1 displayed on the monitor 30 along with his/her work schedule. FIG. 9 shows a behavioral plan for a part of the set period.

In the example shown in FIG. 9, commuting to a workplace is scheduled on April 11, 13 and 15, and telecommuting is scheduled on April 12 and 14. Further, a meeting that will be held at a client company or the like is scheduled in the afternoon of April 15, and a subsequent dinner with the client or the like is also scheduled. In this case, the body shape management system 2 calculates a behavioral plan while also taking calories that will be taken in the dinner, calories that will be consumed by the commuting, and calories that will be consumed by the traveling between the workplace and the client company or the like into consideration. In the behavioral plan shown in FIG. 9, it is proposed to incorporate, as meals, lunch menus M8_1 to M8_7 for seven days and dinner menus M8_8 to M8_13 for six days, i.e., days except for the day on which the dinner with the client or the like will be held, in each week. Note that, for example, menus in a canteen of the own company or the like may be used for the lunch menus. Further, in the behavioral plan shown in FIG. 9, it is proposed to incorporate, as exercise items, swimming in the spare time in the morning of April 10 and running at nights of April 12 and 14. Further, it is proposed to walk from the workplace to the client company or the like and from the client company or the like to the place where the dinner will be held.

In this way, the body shape management system 2 can propose a more suitable behavioral plan without affecting the already fixed schedule of the user U1 and while taking the effect (e.g., the calorie intake and calorie consumption) of the already fixed schedule of the user U1 into consideration.

### Third Embodiment

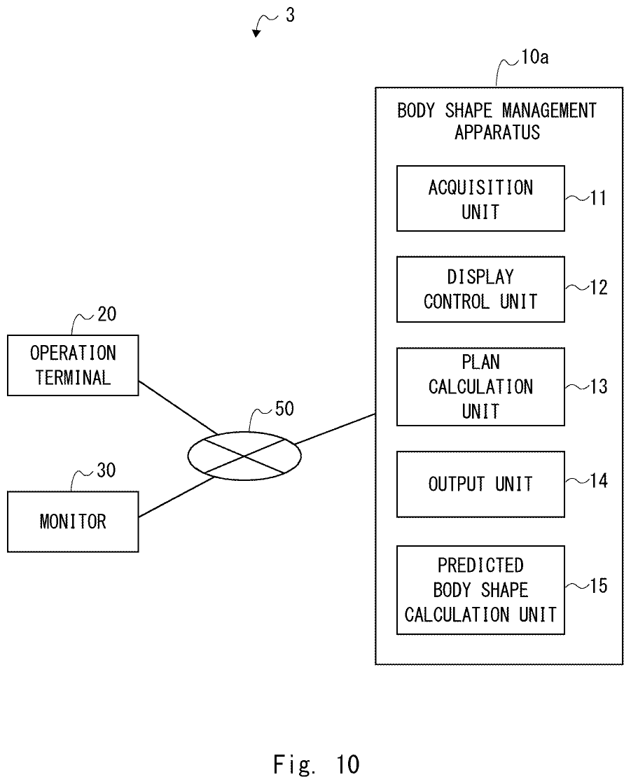

FIG. 10 is a block diagram showing an example of a configuration of a body shape management system 3 according to a third embodiment. The body shape management system 3 according to this embodiment periodically acquires information about the actual body shape of the user U1, calculates a predicted body shape of the user U1 after a lapse of a set period from changes in the acquired actual body shape of the user U1, and displays the calculated predicted body shape on the monitor 30 along with the target body shape. In this way, the body shape management system 3 can visually express the achievement level of the user U1 for the target. Details of the body shape management system 3 or the like will be described hereinafter.

As shown in FIG. 10, the body shape management system 3 includes a body shape management apparatus 10a instead of the body shape management apparatus 10 as compared with the body shape management system 1. The body shape management apparatus 10a further includes a predicted body shape calculation unit 15 as compared with the body shape management apparatus 10. The rest of the configuration of the body shape management system 3 is similar to that of the body shape management system 1, and therefore the description thereof is omitted.

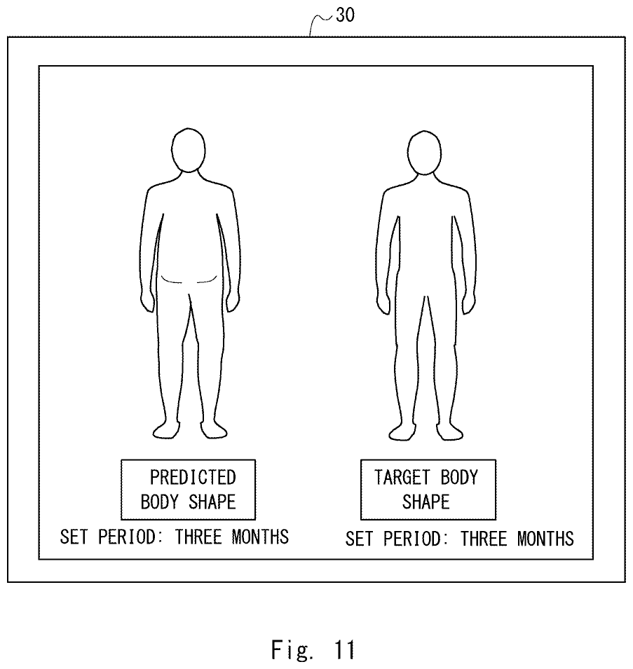

In the body shape management apparatus 10a, the acquisition unit 11 periodically acquires information about the actual body shape of the user U1. The predicted body shape calculation unit 15 calculates a predicted body shape of the user U1 after a lapse of a set period (i.e., a body shape that the user U1 will have after the lapse of the set period) according to changes in the actual body shape of the user U1 which is periodically acquired by the acquisition unit 11. The display control unit 12 further displays, on the monitor 30, the predicted body shape of the user U1 after the lapse of the set period in addition to the target body shape of the user U1 that he/she wants to have after the lapse of the set period. FIG. 11 shows an example of an avatar representing the predicted body shape of the user U1 and an avatar representing the target body shape of the user U1 displayed on the monitor 30. In this way, the user U1 can visually know his/her achievement level for the target from a difference between his/her predicted body shape and his/her target body shape.

Note that in the body shape management apparatus 10a, the plan calculation unit 13 may recalculate at least one plan (at least one behavioral plan) for behavioral items that the user U1 should perform during the set period based on the difference between the predicted body shape and the target body shape.

As described above, the body shape management system 3 according to this embodiment calculates a predicted body shape of the user U1 after a lapse of a set period from changes in the actual body shape of the user U1, and displays the calculated predicted body shape on the monitor 30 along with the target body shape. In this way, the body shape management system 3 can visually express the achievement level of the user U1 for the target, and recalculate the behavioral plan as required.

Note that the present disclosure is not limited to the above-described embodiments, and they can be modified as appropriate without departing from the scope and spirit of the disclosure. Further, the present disclosure may be implemented by combining the above-described embodiments with each other as appropriate.

Further, in the present disclosure, some or all of the control processes performed in the body shape management system can be implemented by having a CPU (Central Processing Unit) execute a computer program.

In the aforementioned program includes instructions (or software codes) that, when loaded into a computer, cause the computer to perform at least one of the functions described in the embodiments. The program may be stored in a non-transitory computer readable medium or a tangible storage medium. By way of example, and not a limitation, the non-transitory computer readable media or tangible storage media can include a RAM (Random-Access Memory), a ROM (Read-Only Memory), a flash memory, an SSD (Solid-State Drive) or other types of memory technologies, a CD-ROM, a DVD (Digital Versatile Disc), a Blu-ray (Registered Trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, the transitory computer readable media or communication media can include electrical, optical, acoustical, or other forms of propagation signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A body shape management system comprising:
an acquisition unit configured to acquire information about an actual body shape of a user and information about a target body shape of the user that he/she wants to have after a lapse of a set period;
a display control unit configured to display at least an avatar representing the target body shape of the user acquired by the acquisition unit on a monitor;
a plan calculation unit configured to calculate at least one plan for a behavioral item that the user should perform during the set period based on the actual body shape of the user and the target body shape of the user that he/she wants to have after the lapse of the set period; and
an output unit configured to output the at least one plan calculated by the plan calculation unit.

2. The body shape management system according to claim 1, wherein
the display control unit displays the avatar representing the actual body shape of the user on the monitor in such a manner that the avatar can be edited, and
the acquisition unit acquires the information about the target body shape of the user that he/she wants to have after the lapse of the set period based on the editing made to the avatar representing the actual body shape of the user displayed on the monitor.

3. The body shape management system according to claim 1, wherein
the acquisition unit is further configured to acquire, in addition to the information about the actual body shape of the user, a predetermined plan for the behavioral item that the user should perform during the set period,
when the predetermined plan is acquired by the acquisition unit, the display control unit displays an avatar representing a predicted body shape of the user on the monitor in such a manner that the avatar can be edited, the predicted body shape being a body shape which it is predicted the user will have after the lapse of the set period on a condition that the user behaves according to the predetermined plan, and
the acquisition unit further acquires the information about the target body shape of the user that he/she wants to have after the lapse of the set period based on the editing made to the avatar representing the predicted body shape of the user displayed on the monitor.

4. The body shape management system according to claim 3, wherein the plan calculation unit gives preference to incorporating an exercise item and a meal included in the predetermined plan into the calculated plan over an exercise item and a meal that are not included in the predetermined plan.

5. The body shape management system according to claim 1, further comprising a database in which a schedule of the user during the set period is stored, wherein
the plan calculation unit calculates at least one plan for a behavioral item that the user should perform during the set period based on, in addition to the actual body shape of the user and the target body shape of the user that he/she wants to have after the lapse of the set period, the schedule of the user during the set period stored in the database.

6. The body shape management system according to claim 1, wherein the display control unit further displays the avatar representing the actual body shape of the user on the monitor.

7. The body shape management system according to claim 6, wherein the display control unit superimposes the avatar representing the actual body shape of the user on the avatar representing the target body shape of the user on the monitor.

8. The body shape management system according to claim 6, wherein the display control unit displays the avatar representing the actual body shape of the user and the avatar representing the target body shape of the user in an alternate manner.

9. The body shape management system according to claim 1, further comprising a predicted body shape calculation unit configured to calculate the predicted body shape of the user after the lapse of the set period according to a change in the actual body shape of the user acquired by acquisition unit, wherein
the display control unit further displays the avatar representing the predicted body shape of the user calculated by predicted body shape calculation unit on the monitor.

10. The body shape management system according to claim 9, wherein the display control unit superimposes the avatar representing the predicted body shape of the user on the avatar representing the target body shape of the user on the monitor.

11. The body shape management system according to claim 9, wherein the display control unit displays the avatar representing the predicted body shape of the user and the avatar representing the target body shape of the user in an alternate manner.

12. The body shape management system according to claim 9, wherein the plan calculation unit recalculates at least one plan for a behavioral item that the user should perform during the set period based on a difference between the predicted body shape and the target body shape.

13. The body shape management system according to claim 1, wherein the output unit displays the at least one plan calculated by the plan calculation unit on the monitor.

14. The body shape management system according to claim 1, wherein the output unit displays a plan selected from the at least one plan calculated by the plan calculation unit along with the schedule of the user on the monitor.

15. A method for controlling a body shape management system, comprising:
acquiring information about an actual body shape of a user and information about a target body shape of the user that he/she wants to have after a lapse of a set period;
displaying at least an avatar representing the acquired target body shape of the user on a monitor;
calculating at least one plan for a behavioral item that the user should perform during the set period based on the actual body shape of the user and the target body shape of the user that he/she wants to have after the lapse of the set period; and
outputting the calculated at least one plan.

16. A non-transitory computer readable storage medium storing a control program for causing a computer to perform:
a process for acquiring information about an actual body shape of a user and information about a target body shape of the user that he/she wants to have after a lapse of a set period;
a process for displaying at least an avatar representing the acquired target body shape of the user on a monitor;
a process for calculating at least one plan for a behavioral item that the user should perform during the set period based on the actual body shape of the user and the target body shape of the user that he/she wants to have after the lapse of the set period; and
a process for outputting the calculated at least one plan.
